(19) United States
(12) Patent Application Publication
(10) Pub. No.: US 2011/0098256 A1
(43) Pub. Date: Apr. 28, 2011

(54) Method for making and using a topical dermatological solution

(76) Inventor: Dixie J. Hines, Hilton Head, SC (US)

(21) Appl. No.: 12/589,371

(22) Filed: Oct. 22, 2009

(51) Int. Cl. A61K 31/60; A61P 17/00

(52) U.S. Cl. 514/159

(57) ABSTRACT

A novel method for making and using topical dermatological solution for external use which is effective in treating dermatological disorders. The methods include administering a therapeutically effective amount of a topical dermatological solution comprising pre-mixed agents which can affect a variety of inflammatory conditions of the skin thus treating a variety of minor skin irritations resulting from an array of causes such as dermatitis, insect bites, skin rash, plant poisoning, acne, pimples, folliculitis, sunburn, burns, and the like. The method for making this unique curative solution includes mixing particular proportions of aspirin, white vinegar, table salt, and baking soda. The present invention further provides a topical dermatological solution and a method for applying the topical dermatological solution to the skin from a single dispensing and applicator sheet, wherein said solution is released from the sheet and applied to the afflicted skin area.

### FIELD OF INVENTION

The present disclosure relates generally to topical dermatological agents for the skin, and more specifically, the present disclosure relates to treating inflammations of the skin with topical solutions.

### BACKGROUND

Many substances are applied topically to the skin of humans or animals (hereinafter “skin”) for therapeutic, preventive or cosmetic purposes. These substances may generically be termed “topical products” and include topically applied substances. Examples of topical products commonly classified as “topical drugs” are many and varied, and include over-the-counter and/or prescription products such as sunburn treatments, anti-acne agents, anti-dandruff agents, external analgesics, topical drug delivery systems, and many other products with therapeutic or other effects.

Also, environmental influences may themselves increase the skin's sensitivity by reducing the skin's “barrier function.” The barrier function acts to minimize absorption or passage of potentially irritating chemicals through the outer “dead” cell layer into the living skin tissue. In some instances, topically-applied products (e.g., soaps, detergents, cleansing products, shaving creams, alcohol and other products which remove some of the skin's protective lipids and/or secretions) may increase the skin's permeability and sensitivity.

Normal processes such as sweating may also increase the ability of irritant materials, such as antiperspirants, deodorants or sunscreens, to penetrate the skin through pores or glands, thus exacerbating the potential for irritation. Exposure of the skin to high humidity environments or liquids may also increase the ability of potential irritants to penetrate the skin. Similarly, the skin may become sensitized or inflamed due to infection, shaving abrasion, repeated or excessive washing or bathing, sun exposure, or other mechanical abrasion or injury, resulting in sensory irritation responses upon subsequent application of underarm deodorants, after-shaves or other topical products.

Moreover, the sensation of itch is one of the most common skin problems experienced by humans and animals. Itch can be defined as a sensation which provokes the desire to scratch the site from which the sensation originates. Skin contains sensory nerves which can transmit itch in response to chemical irritation, environmental exposure or disease processes. Although the precise population of itch producing nerves have not been identified, the thinnest, unmyelinated nerve population, termed type C nociceptive neurons are thought to be the most important in producing the sensation. Itch: Mechanisms and Management of Pruritus, Jeffrey D. Bernhard, McGraw-Hill, Inc. (San Francisco, 1994), pp. 1-22. The itch-producing nerves of the skin can be considered to be a “final common pathway” for the many irritating conditions which are ultimately sensed as itch including chemical exposure, environmental exposure (such as that which produces dry, itchy skin) and disease processes such as atopic dermatitis. Many chemical substances are able to produce itch when topically applied to the skin. Many chemical substances that are currently found in various prescription medicines and other over the counter medicines may also produce irritations or itching. No matter what the ultimate cause of itch, the sensation experienced is the same and provokes the desire to scratch.

Whatever the exact cause of irritation, many attempts have been made to reduce the itch and irritation affects on the skin with various means. U.S. Pat. No. 5,834,409 to Ramanchandran, patented Nov. 10, 1998, entitled Scalp Care Products Containing Anti Itching/Anti Irritant Agents is one such prior art teaching of using aspirin in shampoo as an anti-inflammatory agent. U.S. Pat. No. 5,460,620 to Smith, patented Oct. 24, 1995 is for a Method of Applying In-Tandem Applicator Pads for Transdermal Delivery of a Therapeutic Agent. This patent teaches of a patch-like device for administering medicants to the skin, such as doses of dermatological agents.

### SUMMARY

This disclosure relates to methods and solutions for treating dermatological disorders. The methods include administering a therapeutically effective amount of a topical dermatological solution comprising pre-mixed agents which can affect inflammatory conditions of the skin.

The present invention provides a topical dermatological solution and a method for applying the topical dermatological solution to the skin from a single dispensing and applicator sheet, wherein said solution is released from the sheet and applied to the target area of the skin.

There is a need for simple, but effective means for treating a variety of minor skin irritations resulting from an array of causes such as dermatitis, insect bites, skin rash, plant poisons (e.g., poison ivy/oak) sunburn, burns, and the like.

Also, there is a need for a packaging and delivery system for dispensing such active ingredients, which has a simple configuration for releasing the contents of the packaging without the need to perform any mixture, but provides ready dispensing of the package contents in an easily disposable, and very portable manner.

The present invention provides a novel system for containing, dispensing and essentially simultaneously applying a topical dermatological solution which may contain a plurality of bioactive agents to a target surface, such as the surface of the skin having anti-itch and anti-inflammatory effects, and therefore healing and soothing (i.e., anti-pain) affects.

Moreover, all of the ingredients used to make the topical solution are “relatively natural” and each has proven physiological affects on the skin. This topical solution of the instant invention does not require any preservatives, additives, or other chemicals which many find to be irritating to skin. Importantly, this “off the shelf” combination of household items makes this topical solution very accessible and inexpensive to produce.

These and other advantages of the invention will become more apparent after reading the description and claims which follow.

### BRIEF DESCRIPTION OF THE DRAWINGS

Embodiments of the present disclosure are described herein with reference to the drawings, in which:

FIG. 1 is a perspective view of an embodiment of a dispensing and application device for delivery of the topical dermatological solution as provided by the instant invention;

FIG. 2 is a perspective view of the dispensing and application device for delivery of the topical dermatological solution shown enclosed in a cover envelope, in accordance with an embodiment of the invention; and

FIG. 3 is a perspective view of the dispensing and application device for delivery of the topical dermatological solution shown enclosed in a partially open cover envelope, in accordance with an embodiment of the invention

### DETAILED DESCRIPTION

This application relates to methods and solutions for treating dermatological disorders. The methods include administering a therapeutically effective amount of a topical dermatological solution comprising pre-mixed agents which can affect inflammatory conditions of the skin. In the foregoing description, particular amounts of bioactive ingredients in combination have been found to be effective for an array of inflammatory skin condition ranging from burns to scalp irritations. The particulars of these ingredients and combinations thereof will be discussed as follows. All percentages are by weight unless otherwise indicated. In the present invention, particular amounts of the combination of the ingredients consisting of aspirin, white vinegar, table salt and baking soda, have been found to be an effective topical dermatological solution.

Acetylsalicylic acid, or more commonly known as aspirin, is a white crystalline powder with a melting point of 135° C. Its systematic name is 2-(acetyloxy) benzoic acid and its IUPAC name is 2-acetoxybenzoic acid. The chemical formula of aspirin is C₉H₈O₄

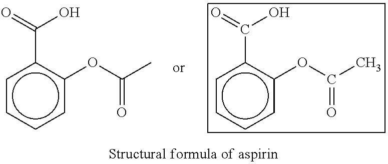

White vinegar is a 5% solution of acetic acid, C₂H₄O₂ (5 grams of acetic acid in 100 grams of total solution, as the % usually means calculated by mass). The systematic or proper chemical name of acetic acid is ethanoic acid. Vinegar is a weak acid produced by the oxidation of alcohol (ethanol) from wines and ciders by bacteria.

The condensed structural formula for acetic acid is CH₃COOH. This formula is a shorthand way of representing the complete structural formula as indicated below:

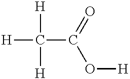

The first carbon atom is bonded to three hydrogen atoms and a second carbon atom. The second carbon atom is bonded to the first carbon atom and to one oxygen atom and an OH group. Hence it's formula is CH₃COOH. The molecular mass or molar mass of acetic acid is 60.05 g/mol.

Salt is an ionic compound. There are many different types of salts. The most common salt is sodium chloride, NaCl or table salt, and is made up of the elements sodium and chlorine. Sodium is an element found in Group I of the Periodic table, the alkali metals. Sodium has a low density which is less than water and is very soft.

The proper chemical name for sodium bicarbonate or baking soda is sodium hydrogen carbonate. Sodium bicarbonate (also dubbed bicarb of soda) is a white powder which is commonly used as an antacid and in cooking as a leavening agent as it reacts with acidic ingredients. It is a very weak base. Bicarb of soda can also neutralize both acids and bases due to its amphoteric nature. The chemical formula for sodium bicarbonate is NaHCO₃ and its molecular structure is indicated by the following:

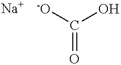

The molecular mass or molar mass of sodium bicarbonate is 84.007 g/mol Baking soda vigorously reacts with vinegar to produce carbon dioxide gas. The chemical equation for the reaction is:

Sodium bicarbonate and vinegar=>Sodium acetate and water and carbon dioxide

NaHCO₃+CH₃COOH═>CH₃COO⁻Na⁺+H₂O+CO₂

In a preferred embodiment of the present invention, an effective topical dermatological solution requires the following percentages in weight comprising:
2.7% of aspirin;
66% of white vinegar;
20.1% of table salt; and
11.2% of baking soda.

Therefore, in one embodiment, a mixture of approximately 4.875 grams of aspirin, approximately 120 grams of white vinegar (½ cup), approximately 36 grams of table salt (2 tbsp.), and approximately 20 grams of baking soda (2 tbsp.) would yield an effective topical dermatological solution for treatment of inflammations of the skin.

When preparing this solution of this embodiment, 4.875 grams of aspirin are provided, which can comprise the step of grinding 15 aspirin of 325 mg strength. This is a common “off the shelf” potency and is easily obtained. This aspirin quantity is added to the vinegar, salt and baking soda and well stirred until thoroughly mixed. This solution should have a shelf life of about 6 months and can be stored in the refrigerator to maintain shelf life. An exemplary refrigeration temperature range is somewhere between 35 and 38 degrees F. (1.7 to 3.3 degrees C.).

Much of the effectiveness of this solution may lie in the ionic strength of the solution. This is because the ionic strength will drive the osmotic pressure toward equilibrium which causes the number of ions inside of a membrane and the number of ions on the outside of a membrane toward unity. Furthermore, both aspirin and vinegar are weak acids and baking soda is a weak base. Salt has no acidity or basicity and is thus a neutral component to the solution. The salt in the solution causes the osmotic pressure to be stronger on the solution side, which would thus promote healing.

Moreover, all of the ingredients used to make the topical solution are “relatively natural” and each has proven physiological affects on the skin. This solution does not require any preservatives, additives, or other chemicals which many find to be irritating to skin. Importantly, this “off the shelf” combination of household items makes this topical solution very accessible and inexpensive to produce.

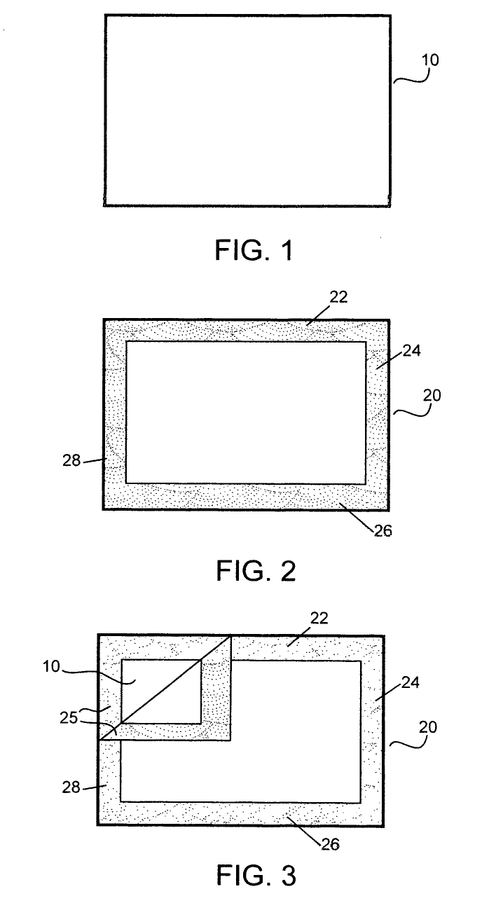

FIGS. 1-3 illustrate exemplary devices for administering the disclosed inventive dermatological topical solution. FIG. 1 illustrates a dispensing and applicator device, and in one embodiment, the dispensing and applicator device may comprise an absorbent sheet designated generally by the numeral 10, saturated with the dermatological agents of the instant invention. The absorbent sheet may serve as a medicated element used to contain, preserve and deliver the solution transdermally. The sheet 10 is intended for topical application to the skin for the treatment of dermatitis for inflammations resulting from burns, sunburns, minor abrasions, insect bites, plant poisons, scalp irritations, rashes, pimples, acnes, folliculitis, and the like. As depicted, dispensing and applicator device may have a generally rectangular configuration. However, it will be understood that a variety of shapes and sizes can be accommodated according to the invention.

FIG. 2 illustrates an enclosure device for the absorbent sheet 10. The enclosure device may take on the form of a cover envelope 20 to encase and enclose the absorbent sheet 10. The cover envelope 20 should be comprised of material relatively puncture resistance, non-absorbent, and impermeable, chemically compatible and non-reactive with the disclosed topical solution contained in the dispensing and applicator device 10 to prevent leakage or migration of the contents out of the dispensing and applicator device 10, and substantially impermeable to external contaminants such as air, dust, liquids and the like.

Suitable materials for the cover envelope 20 should be materials capable of allowing cover envelope 20 to be sealed to itself along seal lines 22, 24, 26 and 28 in order to form a containing and dispensing package that does not separate during normal use. For example, cover envelope 20 may be made of a thermoplastic and heat sealable polymeric film material, such as polyethylene, polyvinyl chloride, or polyamide-type resins, according to known techniques in the art. Such a film may be used alone or adhered to a non-heat sealable material by known techniques. The cover envelope 20 may be formed, for example, of glassine paper, cellophane, polyethylene, polypropylene, polyvinyl chloride, polyester, nylon and the like. Cover envelope 20 may also be formed of an aluminum foil that is coated or sealed with a thermoplastic material such as a polyethylene, polyester, polyvinyl resin or cellulose acetate. Alternatively, the foil may comprise a cellulosic material lined with a thermoplastic film or other synthetic or plastic material. A foil-lined paper board may also be used. A coating to provide a nonporous gas and/or vapor barrier, as for example polyethylene and/or polyester, may be applied to the outer surface of cover envelope 20. The coating may provide a sealant to prevent exposure of the sheet to environment of contamination.

Cover envelope 20 may be sealed to itself by a heat seal, pressure seal, high frequency seal, ultrasonic seal, a crimp, a bonding material or various adhesive materials. Other suitable attachment method or means, may be used to effect a secure seal according to known techniques in the art. For example, heat may be applied according to known techniques in the art to cause a bonding of the thermoplastic liner of cover envelope 20 to itself. A temporary heat seal may be formed by applying relatively narrow lines of heat seal, and wider lines of heat seal may be applied to affect a more permanent seal.

Any adhesives 25 used to form seal lines 22, 24, 26, and 28 should be non-reactive and compatible with the material used for cover envelope 20 as well as with the contents within the absorbent sheet 10, and should not permit premature leakage or diffusion of such materials from the package. An example of adhesives for affecting a releasable seal include, for example, polyvinyl chloride (PVC) applied to one surface, and polyvinyl acetate applied to a second surface. Cover envelope 20 may also be sealed together by a piece of material (not shown) attached between the two surfaces of cover envelope 20.

The dermatological topical solution will be absorbed into a single sheet in an amount effective to provide a unit dosage of each of the bioactive ingredients therein. It is to be understood that such a dose can vary widely, as it will be based on the size of the skin area to be treated, and the nature and severity of the dermatological condition to be treated. The dose may also be varied based on the size of the sheet and the material of which it is constructed and the skin area and condition to be treated. Thus, the amount of active ingredient delivered as a unit dose can be pre-selected, using the weight percentages provided hereinabove as guidelines.

In practice, the user having an affliction of dermatitis would remove an absorbent sheet 10 from its cover envelope 20, expose the medicated absorbent sheet 10 and apply it manually to the afflicted skin area. The dermatological agent solution would then be released onto the skin of the user. It is envisioned that the topical dermatological solution may be used in a healing, curative means for treatment for an array of skin afflictions including the non-exhaustive listing of skin irritations and itching, inflammations, acne, plant poisoning, burns, sunburns, pimples, acnes, folliculitis, and the like.

In the foregoing manner, exemplary embodiments of the present disclosure are described with reference to the figures. Although only exemplary embodiments are of the present disclosure are described, the present invention is not to be limited to specific details so described. The scope of the present disclosure is not limited to the exemplary embodiments of the present disclosure provided above. Numerous changes and modifications can be made to the exemplary embodiments without departing from the scope or spirit of the present invention.

What is claimed is:

1. A solution comprising:
approximately 2.7% of aspirin, approximately 66% of white vinegar, approximately 20.1% of table salt, and approximately 11.2% of baking soda; and

2. The solution of claim 1, wherein,
said solution provides an anti-inflammatory and soothing effect on the skin.

3. The solution of claim 1, wherein,
said solution provides anti-itch effects on the skin.

4. The solution of claim 1, wherein,
said solution provides curative effects for skin afflictions such as dermatitis, insect bites, skin rash, plant poisoning, acne, pimples, acnes, folliculitis, sunburn, burns, and the like.

5. A method for preparing a topical dermatological solution for managing one or more dermatological conditions in skin which comprises:
providing approximately 2.7% of aspirin, approximately 66% of white vinegar, approximately 20.1% of table salt, and approximately 11.2% of baking soda;
adding said aspirin to the white vinegar, table salt, and baking soda; and
mixing said aspirin to the white vinegar, table salt, and baking soda by stirring until thoroughly mixed.

6. The method of claim 5, wherein,
said solution cool is kept cool.

7. The method of claim 5, wherein,
said solution is added to a delivery and dispensing mechanism.

8. The method of claim 7, wherein,
said delivery and dispensing mechanism is an absorbent sheet saturated with said solution.

9. The method of claim 8, wherein,
said absorbent sheet is sealed and contained within a cover envelope.

10. A dispensing and applicator device having a topical dermatological solution comprising a mixture consisting of:
approximately 2.7% of aspirin, 66% of white vinegar, 20.1% of table salt, and 11.2% of baking soda,
wherein said solution provides an anti-itch, anti-inflammatory and soothing effect on the skin

11. The dispensing and applicator device of claim 10, further comprising
a medicated absorbent sheet which is saturated with said solution.

12. The dispensing and applicator device of claim 10, further comprising
a cover envelope for encasing said medicated absorbent sheet.
